(19) United States
(12) Patent Application Publication
(10) Pub. No.: US 2021/0178353 A1
(43) Pub. Date: Jun. 17, 2021

(54) NUCLEIC ACID IMMOBILIZATION ARTICLE AND METHODS THEREOF

(71) Applicant: CORNING INCORPORATED, CORNING, NY (US)

(72) Inventors: Jeffrey Glenn Lynn, Wellsboro, PA (US); Shawn Michael O'Malley, Horseheads, NY (US)

(21) Appl. No.: 16/759,863

(22) PCT Filed: Oct. 30, 2018
(86) PCT No.: PCT/US2018/058195

§ 371 Date: Apr. 28, 2020

Related U.S. Application Data
(60) Provisional application No. 62/578,798, filed on Oct. 30, 2017.

(51) Int. Cl. B01J 19/00; C12Q 1/6874

(52) U.S. Cl. CPC B01J 19/0046; C12Q 1/6874; B01J 2219/00529; B01J 2219/0063; B01J 2219/00608; B01J 2219/00632; B01J 2219/00722; B01J 2219/00626

(57) ABSTRACT

An article including: a substrate; and at least one immobilization site on the substrate comprising at least one nucleic acid immobilization site, each site having a first layer of a tie agent in contact with the substrate, and a second layer of a dendrimer mobilizing agent in contact with the first layer. Also disclosed is a method of making and a method of using the article.

### CROSS-REFERENCE TO RELATED APPLICATIONS

This application claims the benefit of priority under 35 U.S.C. § 119 of U.S. Provisional Application No. 62/578,798, filed Oct. 30, 2017, the content of which is incorporated herein by reference in its entirety.

The present application is related commonly owned and assigned U.S. Provisional Patent Application Ser. No. 62/559,951, entitled “FLOW CELLS HAVING REACTIVE SURFACES FOR NUCLEIC ACID SEQUENCE ANALYSIS”, filed Sep. 18, 2017, but does not claim priority thereto.

The entire disclosure of each publication or patent document mentioned herein is incorporated by reference.

### BACKGROUND

The disclosure relates to an article and a method for making the article having a patterned DNA immobilization surface.

### SUMMARY

In embodiments, the disclosure provides an article and a method for making the article having a patterned nucleic acid (e.g., DNA) immobilization surface.

In embodiments, the disclosure provides a method for using the article for nucleic acid (e.g., DNA) immobilization, for example, in a flow cell.

### BRIEF DESCRIPTION OF THE DRAWINGS

In embodiments of the disclosure:

FIG. 1 shows a sequential coating procedure for making the DNA immobilizing NH₂ PAMAM generation-5 dendrimer on a GLYMO surface.

FIG. 2 shows a 2D and a 3D representation of branching of a dendrimer structure.

FIG. 3 show an example of a second generation (G=2) polyamidoamine dendrimer from Dendritech.

FIG. 4 shows how a disclosed patterned high nucleic acid or DNA binding chemistry can be used to do genomic next gen sequencing.

FIG. 5 shows a flow cell experiment comparing DNA retention after 12 hrs of extensive continuous flow of buffer (65° C.) on four different surface chemistries.

FIG. 6 is a bar chart showing the amount of nucleic acid retention as measured by relative fluorescence counts using Sytox orange DNA stain for four different surface treatments.

FIGS. 7A and 7B show images of the same sample having slightly different confocal brightness, which images demonstrate selective photolithographic patterning using the GLYMO/NH₂ dendrimer coating method.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

### Definitions

“Include,” “includes,” or like terms means encompassing but not limited to, that is, inclusive and not exclusive.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, times, and like aspects, and ranges thereof, are for illustration only; they do not exclude other defined values or other values within defined ranges. The composition and methods of the disclosure can include any value or any combination of the values, specific values, more specific values, and preferred values described herein, including explicit or implicit intermediate values and ranges.

In embodiments, the present disclosure provides an article comprising:

a substrate; and

at least one immobilization site on the substrate comprising at least one nucleic acid, e.g., DNA, RNA, and like molecules, immobilization site, each site having a first layer of a tie agent in contact with the substrate, and a second layer of a dendrimer immobilizing agent in contact with the first layer, i.e., the virgin product.

In embodiments, the dendrimer immobilizing agent can be, for example, a dendrimer molecule terminated with at least one of, for example: an —NH₂, an —NRH, an —NH₃⁺, an —NRH₂⁺, an —NR₂H⁺, an —NR₃⁺, or a combination thereof, where R is, for example, an (C₁ to C₁₀)alkyl.

In embodiments, the article can further comprise, for example, at least one immobilized nucleic acid on at least one nucleic acid immobilization site, i.e., the DNA or RNA reacted or pre-reacted product suitable for interaction with, e.g., other DNA, RNA, proteins, etc.

In embodiments, the at least one immobilization site can be selected, for example, from a single site, a plurality of sites, an array pattern, an entire surface area of one major side of the substrate.

In embodiments, the array pattern can comprise, for example, a plurality of the nucleic acid immobilization sites having a size of from 5 to 1,000 nm, e.g., 5 to 800 nm, 100 to 1,000 nm, 5 to 50 nm for example, by e-beam, and each site can be separated from a neighboring site by, for example, of from 100 to 500 nm.

In embodiments, the substrate is at least one of a low auto-fluoresence material selected from, for example, a transparent glass, a transparent glass-ceramic, a transparent ceramic, a transparent plastic, and like materials, or a combination thereof.

In embodiments, the tie agent can be, for example, a CVD coatable silane, e.g., an epoxysilane such as GLYMO, which does not block the photoresist etch step, and the dendrimer immobilizing agent can be, for example, an amine terminated, star branching dendrimer having a spheriodal 3D geometry or shape, and from 3 to 10 propagation generations, e.g., polyamindoamine (PAMAM) dendrimer from Dendritech.

In embodiments, the substrate can have a thickness, for example, of from 100 to 500 microns, e.g., 150 to 350 microns, 170 to 300 microns, including intermediate values and ranges. A thinner substrate is preferable to a thicker substrate because of superior optical imaging from the same or the opposite side of the unbound or nucleic acid bound article, and reduced material costs.

In embodiments, the article can further comprise a flow cell having a chamber that surrounds at least one article.

In embodiments, the flow cell can have an inlet and an outlet, and the at least one article can include a plurality of articles.

In embodiments, the present disclosure provides a method for making the abovementioned article, comprising:

coating a substrate with a photoresist adhesion promoter, such as HMDS, to form an adhesion promoted substrate;

completely coating the adhesion promoted substrate with a photoresist, e.g., by spin coating, and developing, e.g., by selective light exposure, the resulting photoresist coated substrate to from a selected pattern;

removing the residual photoresist adhesion promoter, e.g., 02 plasma or UV/03 to remove residual promoter, i.e., HMDS; and

developing the selected pattern with a photoresists etchant to produce an etched patterned substrate;

selectively coating the etched patterned substrate by chemical vapor deposition (CVD) with a tie agent, for example, an epoxysilane GLYMO, in the etched areas to form a tie agent coated etched patterned substrate; and either:

removing the photoresist from the tie agent coated etched patterned substrate, e.g., washing with acetone or other suitable solvent, and then contacting the tie agent coated etched patterned substrate with a dendrimer, e.g., a GLYMO coated and patterned glass is immersed into a dendrimer solution at alkaline pH and reacted for about 1 hr, to form a patterned nucleic acid immobilization surface;

or

contacting the tie agent coated etched patterned substrate with a dendrimer and then removing the photoresist from the tie agent coated etched patterned substrate to form a patterned nucleic acid immobilization surface, i.e., the removing and the contacting can be accomplished in regular order or reverse order, i.e., interchangeable.

In embodiments, the method can further comprise a separable support for the substrate, and can further comprise the step of removing the separable support for the substrate, at any time after removal of the photoresist, e.g., a separable silicon wafer.

In embodiments, the separable support can be removed after the chemical vapor deposition (CVD) of the tie agent.

In embodiments, the separable support can have a thickness, for example, of from 100 to 1,000 microns.

In embodiments, the present disclosure provides a method for using the abovementioned article, comprising:

contacting the article with a source of nucleic acid.

In embodiments, the method of using can further comprise analyzing the article for nucleic acid immobilization from the source of nucleic acid.

In embodiments, the present disclosure is advantaged in several aspects including, for example: the method of making permits discrete high precision patterning of the DNA immobilization chemistry; the method of making can be accomplished on any glass surface (glass is advantageous for confocal microscopy applications where low autofluorescence is called for to maximize the signal obtained through DNA sequencing); and the method of making can be combined with chemical crosslinking to increase DNA retention during multiple extended flow cycles.

In embodiments, the disclosure provides a method of making a patterned surface, such as on the bottom of a flow cell, the patterned surface having a potent electrostatic immobilizing polymer structure attached to the surface. The disclosed method permits facile patterning of a glass surface because the method does not block the removal of a photoresist.

The disclosed method uses a CVD coating of an epoxy silane, such as GLYMO, over a photolithographically patterned glass substrate. Once the epoxy silane coating is immobilized on the surface the surface then be chemically reacted with, for example, a molecule having a high amine content, such as a PAMAM dendrimer in, for example, a reactive alkaline buffer. In a flow cell form factor, the modified surface can then be easily stripped of excess photoresist and dried for use in electrostatic immobilization of DNA such as for genomic sequencing. The disclosed dendrimer coated flow cells were evaluated and described herein.

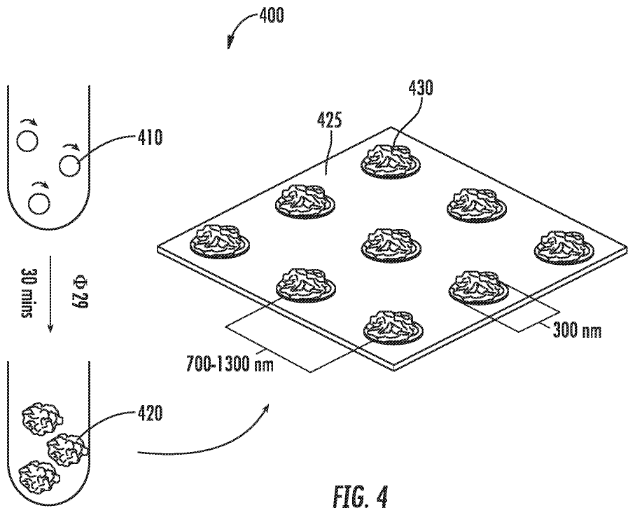

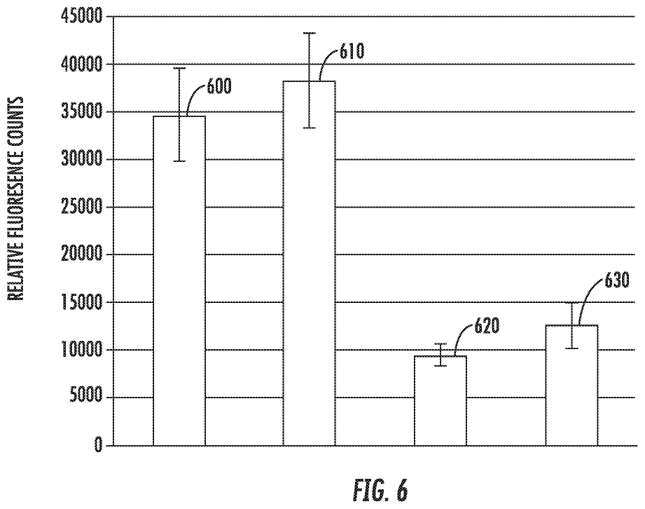

In embodiments, the disclosure provides a method of making a photolithographically patterned immobilization site. In embodiments, the disclosure uses an amine terminated dendrimer to immobilize DNA. The purpose of the dendrimer coating is to provide small such as 500 nm, or smaller, regions or “patches” or “islands” alone or in an array, which region(s) can robustly immobilize DNA (see FIG. 4. DNA nanoballs are a significant target for the disclosed coating and article although essentially any DNA or nucleic acid can be immobilized. The disclosed immobilization surface was tested against the standard low NH₂ content display surface APTES. FIG. 6 shows that the NH₂ PAMAM gen. 5 dendrimer coated over a flat unpatterned GLYMO surface performed well compared to an APTES solution coating for retention of DNA nanoballs after 12 hrs of challenging continuous flowing buffer set to 65° C. as measured by relative fluorescence counts. The APTES was not able to be used for patterned chemistry using a photoresist because the APTES apparently reacted with the photoresist and blocked the removal of the photoresist. Although not limited by theory, it is believed that the single stranded DNA nanoballs (derived from linear (DNA) rolling circle amplification; “L-RCA”) can be retained on the GD CVD surface using electrostatic surface interactions. In embodiments, one can alternatively or additionally modify the dendrimer—nucleic acid retention surface by treating the dendrimer surface with a chemical crosslinking agent such as bis(sulfosuccinimidyl)suberate (BS3).

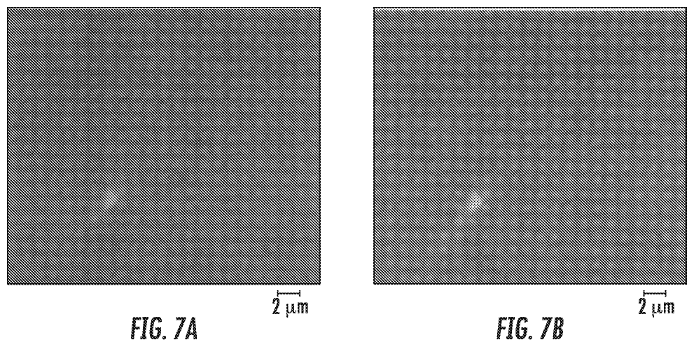

FIGS. 7A and 7B show selective photolithographic patterning was obtained by using the GLYMO/NH₂ dendrimer coating method. FIG. 7A (left side) shows the patterned GD CVD microarray (900 nm spots) that was stained using an amine reactive fluorescent dye ALEXA 555 (i.e., ThermoFisher Scientific Alexa Fluor® 555 NHS Ester (succinimidyl ester)). FIG. 7B (right side) shows the same sample of FIG. 7A but using a slightly higher confocal brightness level. This same specific patterning could not be obtained using APTES because the APTES reacted with the photoresists and blocked its removal.

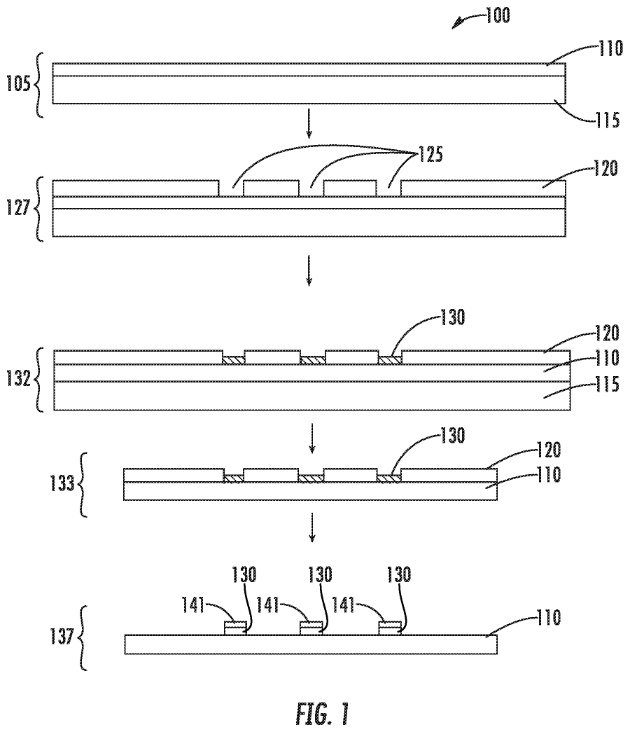

FIG. 1 shows an overview of a sequential method for the manufacture of a photolithographically patterned DNA immobilizing article. Preparative steps for the article can include:

a thin substrate can be selected such as Willow glass, and optionally the substrate can be supported by a removal material such as silicon;

the substrate can be cleaned and coated with a photoresist adhesion promoter HMDS;

the photoresist can be spin coated over the HMDS;

the photoresist can be selectively exposed to light to develop a selected pattern;

the pattern can then be developed using a photoresist etchant;

the resulting patterned substrate can be CVD coated with, for example, an epoxysilane such as GLYMO;

the GLYMO coated patterned glass can then be immersed into a solution containing a dendrimer at alkaline pH and reacted for about 1 hr;

the resulting patterned GLYMO-dendrimer surface can be fully developed by soaking the article or a part in a photoresist stripping solution leaving an HMDS interstitial pattern between the GLYMO dendrimer spots; and

the part or article was washed with water and dried for flow cell bonding and completion.

Referring to the Figures, FIG. 1 shows a schematic (100) representing a sequential coating procedure for making the photolithographically patterned nucleic acid (e.g., DNA) immobilizing NH₂ PAMAM generation-5 dendrimer on a GLYMO surface (137). In a first step, a thin flexible glass substrate (110) such as Willow® glass from Corning Inc., and having a thickness of e.g., 200 microns, is optionally fixed onto a thicker silicon carrier wafer (115) by, for example, electrostatic bonding to support the glass article (105). A thin glass substrate having a thickness of about 170 to 250 microns and having low autofluorescence is preferred for maximizing the signal-to-noise ratio during some sequencing by synthesis applications. Attaching the thin glass to a silicon carrier can be accomplished so that when a vacuum is applied the carrier can reduce the warping of the thin glass. In a second step, the thin glass-silicon wafer combination is 02 plasma cleaned (e.g., 100 W at 300 mTorr for 60 s) and an adhesion promoting silane layer such as HMDS is coated onto the wafer using, for example, a CVD coating chamber system such as a YES Engineering, Inc., silane coater. Then a photoresist such as SPR 955-0.9 from Shipley is spin coated over the HMDS. A photo-pattern is processed using a lithographic stepper tool (common examples include ASML 5500 DUV, GCA 200 i-line, or GCA200). In a third step, the patterned wafer is developed by etching the wafer and exposing the open pattern on the glass to from pattern layer (120) having etched open patterns (130) to produce article (127). To ensure silane coating within the open features within the photoresist the wafer is then subsequently O₂ plasma treated and then CVD coated with an amine reactive silane such as GLYMO to form GLYMO filled resist pattern of the resulting article (32). In a fourth step, the silicon support (115) on the back of the thin glass substrate is then electrostatically debonded from the wafer. In a fifth step, the photoresist (120) is removed with a stripping agent, for example, acetone with continuous sonication for 5 mins, followed by continuous sonication in isopropanol for 5 mins followed by an ethanol rinse and drying (135). The wafer is then solution coated with NH₂ PAMAM dendrimer for 1 hr at 25° C. at a concentration of 50 mg/mL in a 50 mM sodium phosphate buffer, pH 8.3. Following the dendrimer coating, the wafer is rinsed twice in distilled water, acetone, isopropanol, and then dried to produce article (137) having a thin glass substrate (120), and islands of GLYMO supporting the dendrimer (141) on the substrate.

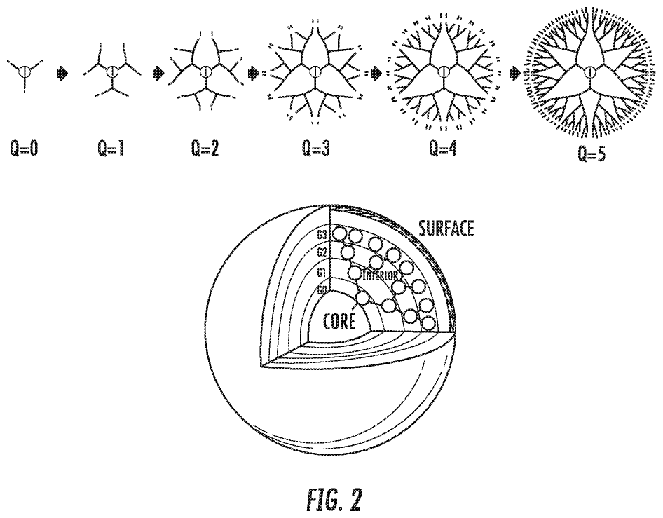

FIG. 2 shows a 2D and a 3D representation of branching of a dendrimer structure. The radial display is of special importance for the electrostatic attraction of DNA structures. The compact spherical structure is also very favorable for packing onto a patterned GLYMO surface. FIG. 2 shows a scheme where in about 5 steps one can obtain the disclosed high DNA binding patterned surface. In the disclosed procedure the glass thickness was too thin to properly develop by photoresist exposure on an I-line system so a silicon carrier wafer was used to stabilize the substrate for photolithography.

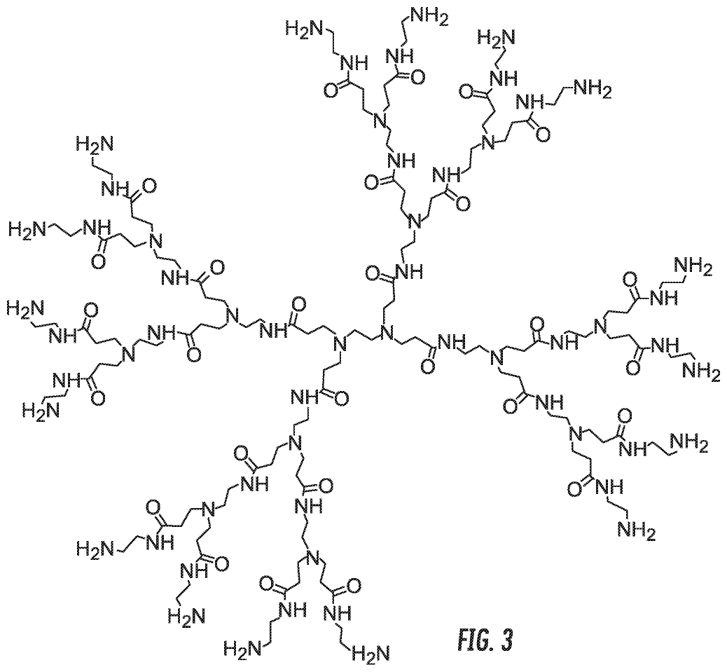

FIG. 3 show an example of a second generation (G=2) polyamidoamine dendrimer from Dendritech.

FIG. 4 shows how a disclosed patterned high nucleic acid or DNA binding chemistry can be used to do genomic next gen sequencing. For example, the genome of an organism is fragmented into short dsDNA strands, which are then made into circular DNA and then amplified by linear rolling circle DNA amplification (L-RCA) technique to yield a large 300 nm repeat copy of a single stranded (ss) genomic DNA. The RCA amplicon is then captured on a surface of patterned flow cell having surface modified chemistry as disclosed herein (see Drmanac, et. al., Human Genome Sequencing using Unchained Base Reads on Self-Assembling DNA Nanoarrays, Science (2010) 327, 78-81).

FIG. 4 shows an example of a possible nucleic or DNA target for immobilization on the patterned NH₂ dendrimer coated glass (see Drmanac, R. supra.). The sequential coating procedure (400) includes a first, amplifying a DNA of interest using a rolling circle DNA amplification (RCA) (410) method to produce a single stranded (ss) genomic DNA; and second, capturing the resulting RCA amplicon (420) on a patterned substrate (425) having active islands of GLYMO surmounted by dendrimer, and surmounted by bound DNA nanoballs (DNB) (430) in a flow cell (not shown). Briefly, genomic DNA is fragmented into 400 nucleotide (nt) regions that are ligated to an adapter that converts the fragments into mini-circular DNA (410). The mini-DNA circles are then primed with a 20 mer compliment oligo and are then amplified into single stranded DNA nanoballs (DNB) which are then immobilized onto a 300 nm patterned NH₂ dendrimer array. Each DNB is an exact copy of the 400 nt region but is amplified by repeated circular copy. When sequencing by synthesis is completed each DNB yields multiple points of fluorescently labelled DNA extension.

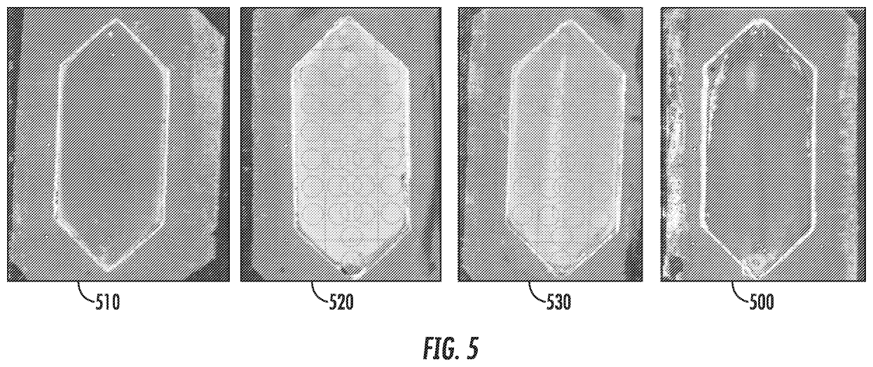

FIG. 5 shows a flow cell experiment comparing DNA retention after 12 hrs of extensive continuous flow of buffer (65° C.) on four different surface chemistries specified below. The immobilized DNA was made visible with a fluorescent stain SYTOX orange dye. The DNA was from a commercial source and was comprised of billions of DNA nanoballs made by linear rolling circle DNA amplification. In the micrographs of FIG. 5 the four surface chemistries were each separately applied to an Eagle XG® glass substrate, including: APTES (3-triethoxysilylpropylamine) coated by solution method (510); APTES CVD deposited by a chemical vapor method (520); APS (aminopropylsilsesquioxane) by solution method (530); and the presently disclosed GLYMO Dendrimer Chemical Vapor Deposition (“GD CVD”) method (500) (where GD stands for “GLYMO Dendrimer” and CVD stands for Chemical Vapor Deposition). The GD CVD method is also referred to as: a GLYMO silane coated by CVD followed by coating with a NH₂ PAMMA dendrimer (Gen 5).

Referring to FIG. 6, the bar chart shows the amount of Sytox orange stained DNA surface retention as measured by relative fluorescence counts for four different surface treatments. The two best surfaces for retention of calf thymus DNA were the GD CVD (600) and the APTES solution coating (610). The APTES CVD coating (620) had a fluorescence of less than 1,000 counts, and the APS coating (630) had a fluorescence of less than 1,250 counts. However, only the GD CVD coating (600) was viable for photolithographic patterning as shown in FIG. 7. The cloud-like smudge regions are imaging artifacts.

In embodiments, the disclosure provides a chemically modified surface and a method for making the chemically modified surface. The chemically modified surface can immobilize nucleic acid such as DNA via electrostatic attraction with the chemically modified surface.

In embodiments, the disclosure provides an article having a chemically modified surface and a method of immobilizing DNA on the modified surface of the article.

In embodiments, the method for making the chemically modified surface includes, for example, a two-step patterning sequence with an NH₂-PAMAM dendrimer or other chemistries having a large array of amine reactive groups (e.g., polyethylenimine). The method includes: coating a glass surface (+/−photoresist) with an amine reactive silane such as GLYMO (3-glycidyloxypropyl) trimethoxysilane, CAS Number 2530-83-8); and immersing a CVD GLYMO coated glass into a slightly alkaline solution of a PAMAM NH₂ dendrimer. The GLYMO provides a tie-layer, which covalently binds to a PAMAM NH₂ dendrimer. The PAMAM NH₂ dendrimer can be, for example, on a flat surface, located in a discrete photolithographic pattern of high density regions on a surface using restrictive pattern photolithography. The disclosed method can make a dense patterned array of PAMAM NH₂ dendrimer regions on a surface using a spherical shaped amine containing polymer.

Effective immobilization of DNA onto a microfluidic surface is significant in numerous genomic applications. Often in genomics one uses a flow cell to sequence genomic DNA by sequentially using a DNA polymerase to add fluorescent DNA bases directly to immobilized DNA on the surface of a flow cell. There numerous ways to immobilize DNA including electrostatic, covalent, hydrophobic, DNA sequence hybridization, and affinity based methods.

In embodiments, the presently disclosed method provides an immobilization surface that can robustly electrostatically immobilize DNA. The immobilization surface can be used, for example, in a microfluidic device, which device can be used, for example, to sequence DNA.

Dense arrays of NH₂ groups are of special relevance for electrostatically immobilizing millions or billions of DNA target molecules, which immobilized molecules can then be sequenced. In embodiments, the presently disclosed method provides solutions to at least two problems related to making the high density arrays.

A first solution is a method of making that uses a silane species (e.g., GLYMO) that does not strongly interact with photoresists (e.g., JSR AR1682J & MEGAPOSIT™ SPR™3000 were tested). It was discovered that some silane coatings can obstruct removal and patterning of a photoresist. During preparing patterned chemical surfaces it was discovered that CVD deposition of APTES (GAPS) over the two photoresists mentioned above can make it impossible to remove the photoresist afterward by using an acetone and ultrasonic bath. Significantly, the GLYMO CVD deposition does not block acetone stripping of the photoresist.

A second disclosed solution is a method of making that yields a very robust surface for electrostatic attraction of nucleic acid by providing a radial array or presentation of amine groups (e.g., —NH₂), ammonium groups (e.g., —NH₃⁺), and like amine or ammonium groups, or a mixture thereof depending on the pH, and the position where the nucleic acid target needs to be located.

In embodiments, the presently disclosed method of making can be accomplished, for example: including a first coating a photolithographically patterned substrate with an epoxy silane such as GLYMO. The GLYMO coating can be, for example, CVD or liquid based. A second step can include, for example, removing the photoresist from the GLYMO treated surface by immersing the surface in an acetone bath followed by ultrasonic treatment. After ultrasonic treatment the resulting surface, part, or article, can be washed such as in ethanol several times, to clean away any loosely bound photoresist. A third step can include, for example, exposing the resulting stripped and washed surface, part, or article, to an aqueous solution containing a dendrimer, such as a PAMAM NH₂ dendrimer. After an incubation period the surface or part is removed and then rinsed in water and ethanol until clean then dried. The resulting dendrimer treated and incubated coated part can then optionally be bonded to another glass part or like pieces to form a completed flow cell.

The dendrimer polymer can be prepared by, for example, known spherically growth methods (see for example, U. Boas, et al., “Dendrimers: Design, Synthesis and Chemical Properties,” Chapter 1 in Dendrimers in Medicine and Biotechnology: New Molecular Tools, 2006 28 pages, ISBN: 978-0-85404-852-(springer.com)). Such repetitively grown branched polymers can have a very high three dimensional radial array of terminal end groups (see Table 1 of the present disclosure, which lists the theoretical number of end groups that correspond to a generation in growth). Dendron polymers are another related class of hyper-branching polymers that can be selected, alternatively or additionally, to the dendrimer polymer immobilizer demonstrated in a working example herein.

Table 1 (from Dendritech.com) lists how a branched dendrimer with amine terminations can yield a uniform mono-dispersed display of amine groups. Primary and secondary terminal groups or primary and secondary end groups, such as primary and secondary amines, are more preferred compared to tertiary or quaternary terminal groups.

In the present disclosure a generation-5 dendrimer having 128 amine groups was prepared or obtained commercially. When this dendrimer polymer was covalently bonded to a GLYMO surface of a patterned glass it yielded a high radial display of amine groups. In embodiments, a NH₂ PAMAM gen-5 dendrimer was coated onto a 500 nm diameter spot array of GLYMO. Although not limited by theory there can be, for example, about 92 dendrimers within each 500 nm GLYMO spot.

TABLE 1
Theoretical number of end groups for each growth generation.^{1.}

| | Molecular | Measured | Surface |
|---|---|---|---|
| Generation | Weight | Diameter (Å) | Groups |
| 0 | 517 | 15 | 4 |
| 1 | 1,430 | 22 | 8 |
| 2 | 3,256 | 29 | 16 |
| 3 | 6,909 | 36 | 32 |
| 4 | 14,215 | 45 | 64 |
| 5 | 28,826 | 54 | 128 |
| 6 | 58,048 | 67 | 256 |
| 7 | 116,493 | 81 | 512 |
| 8 | 233,383 | 97 | 1024 |
| 9 | 467,162 | 114 | 2048 |
| 10 | 934,720 | 135 | 4096 |

^{1.}From Dendritech.com

Historically the dendrimer molecule has been viewed as an early form of nanotechnology where the molecule has a controlled size and charge distribution. The use of the high radial display of the dendrimer to electrostatically attract and precipitate DNA has been extensively studied. One example was a PAMAM NH₂ dendrimer that was used to precipitate DNA as a potent transfection agent (see, e.g., Eichman, et. al., “The use of PAMAM dendrimers in the efficient transfer of genetic material into cells,” PSTT Vol. 3, No. 7 Jul. 2000). Another publication describes methods for coating an entire surface with a uniform distribution of dendrimers (see Stancu, I-C, in SPR Imaging Label-Free Control of Biomineral Nucleation, Intelligent and Biosensors, Book, V. S. Somerset, ed., ISBN 978-953-7619-58-9, pp. 386, January 2010, INTECH, Croatia, available on sciyo.com).

The presently disclosed method differs from the prior reported methods by using a patterned photo-lithographically derived photoresist pattern on glass, which pattern is called for to achieve electrostatic immobilization DNA. The presently disclosed method discovered that GLYMO can be used as an effective tie layer, which allows the PAMAM NH₂ dendrimer to locate onto the patterned surface but does not block the chemical treatment necessary to strip the photoresist off the surface. A preferred dendrimer functional group selection in the present disclosure is a primary amine because the end task involves DNA precipitation. However, in embodiments, the dendrimer can optionally be end terminated with one or more of a variety of alternative or additional functional groups such as —OH, —COOH, —SH, anhydride, nitrile, imine, His-tag binders such as nickel or cobalt, chelators such as nitrilotriacetic acid (NTA), peptides, biotin, alkyne, alkyl halide, ether, ketone, aldehyde, amide, and like functionality, or mixtures thereof. These other functional groups or combinations may require modification to the specific silane tie-agent chemistry.

### EXAMPLES

The following Examples demonstrate making, use, and analysis of the disclosed article and methods in accordance with the above general procedures.

### Example 1

Method of Making the Article Preparative steps for making the article include, for example:

the substrate is cleaned and coated with a photoresist adhesion promoter HMDS;

the photoresist is spin coated over the HMDS;

the photoresist is exposed to light to develop a selected pattern;

the pattern is developed using a photoresist etchant;

the patterned substrate is CVD coated with, for example, an epoxysilane GLYMO;

the GLYMO coated patterned glass is then immersed into a dendrimer solution at alkaline pH and reacted for about 1 hr;

the patterned GLYMO-dendrimer surface is then fully developed by soaking the article or part in a photoresist stripping solution leaving an HMDS interstitial pattern between the GLYMO dendrimer spots; and the part or the article was washed with water and dried for flow cell bonding and completion.

### Example 2

Method of Using the Article. The patterned glymo-dendrimer glass surface prepared in Example 1 can be incorporated, that is, bonded into a flow cell that has an inlet and outlet port allowing multiple cycles of fluid exposure and processing. The assembled flow cell allows one to inject into the channel genomic DNA nanoballs (spherical redundant copies of genomic regions) that will electrostatically attach to the glymo-dendrimer patterns on the glass. Optical fluorescent imaging of the patterns is done as one passes DNA polymerase along with fluorescently labelled dNTPs which sequentially allow whole genomic sequencing by synthesis.

The disclosure has been described with reference to various specific embodiments and techniques. However, it should be understood that many variations and modifications are possible while remaining within the scope of the disclosure.

What is claimed is:

1-16. (canceled)

17. A method comprising:
coating a substrate with a photoresist adhesion promoter to form an adhesion promoted substrate;
coating the adhesion promoted substrate with a photoresist and developing the photoresist by selective light exposure to form a selected pattern;
developing the selected pattern with a photoresist etchant to form an etched patterned substrate;
selectively coating etched areas of the etched patterned substrate by chemical vapor deposition with a tie agent to form a tie agent coated etched patterned substrate;
removing the photoresist from the tie agent coated etched patterned substrate; and
contacting the tie agent coated etched patterned substrate with a dendrimer, either before or after removing the photoresist from the tie agent coated etched patterned substrate, to form a patterned nucleic acid immobilization surface.

18. The method of claim 17, wherein the tie agent comprises (3-glycidyloxypropyl) trimethoxysilane (GLYMO).

19. The method of claim 17, wherein the dendrimer comprises a polyamidoamine (PAMAM) dendrimer.

20. The method of claim 17, wherein the photoresist adhesion promoter comprises hexamethyldisilazane (HMDS).

21. The method of claim 17, comprising immobilizing a plurality of DNA nanoballs on a corresponding array of patches of the patterned nucleic acid immobilization surface.

22. The method of claim 21, wherein the immobilizing the plurality of DNA nanoballs comprises electrostatically immobilizing the plurality of DNA nanoballs on the corresponding array of patches of the patterned nucleic acid immobilization surface.

23. The method of claim 21, wherein each patch of the array of patches of the patterned nucleic acid immobilization surface is 500 nm or smaller.

24. The method of claim 17 comprising removing the substrate from a separable support any time after removal of the photoresist.

25. The method of claim 24 wherein the removing the separable support is performed after the selectively coating the etched areas of the etched patterned substrate by chemical vapor deposition with the tie agent.

26. The method of claim 24 wherein the separable support has a thickness of 100 to 1,000 microns.

27. An article comprising:
a substrate;
a plurality of nucleic acid immobilization sites, each site comprising a dendrimer immobilizing agent coupled to the substrate via a tie agent;
wherein each site has a size of 5 to 1,000 nm, and each site is separated from a neighboring site by 100 to 500 nm;
wherein the dendrimer immobilizing agent comprises a polyamidoamine (PAMAM) dendrimer; and
wherein the tie agent comprises (3-glycidyloxypropyl) trimethoxysilane (GLYMO).

28. The article of claim 27 further comprising an immobilized nucleic acid on the nucleic acid immobilization site.

29. The article of claim 28, wherein the immobilized nucleic acid comprises a DNA nanoball electrostatically immobilized on the nucleic acid immobilization site.

30. The article of claim 27 wherein the plurality of nucleic acid immobilization sites comprises an array pattern.

31. The article of claim 27 wherein the substrate comprises a low auto-fluorescence material selected from a transparent glass, a transparent glass-ceramic, a transparent ceramic, a transparent plastic, or a combination thereof.

32. The article of claim 27 wherein the substrate has a thickness of 100 to 500 microns.

33. The article of claim 27, wherein the article comprises a flow cell comprising a chamber that surrounds the plurality of nucleic acid immobilization sites.

34. The article of claim 33 wherein the flow cell comprises an inlet and an outlet in fluid communication with the chamber.

35. A method for using the article of claim 27 comprising:
contacting the article with a source of nucleic acid.

36. The method of claim 35 further comprising analyzing the article for nucleic acid immobilization from the source of nucleic acid.
